(19) United States
(12) Patent Application Publication
(10) Pub. No.: US 2024/0419238 A1
(43) Pub. Date: Dec. 19, 2024

(54) SYSTEMS AND METHODS FOR PREDICTING AND PREVENTING FAILURE TO RESCUE EVENTS

(71) Applicant: Ohio State Innovation Foundation, Columbus, OH (US)

(72) Inventors: Paula Garvey, Columbus, OH (US); Stephanie Justice, Columbus, OH (US)

(21) Appl. No.: 18/674,000

(22) Filed: May 24, 2024

Related U.S. Application Data
(60) Provisional application No. 63/468,651, filed on May 24, 2023.

(51) Int. Cl. G06F 3/01; G16H 20/00; G16H 50/20; G16H 50/70

(52) U.S. Cl. CPC G06F 3/01; G16H 20/00; G16H 50/20; G16H 50/70

(57) ABSTRACT

According to one aspect, the present disclosure relates to a computer-implemented method to prevent a failure to rescue event. The computer-implemented method includes providing a user with a first query; receiving a first user input in response to the first query; providing the user with a second query, where the second query is at least partially based on the first user input; receiving a second user input in response to the second query; analyzing at least one of the first user input and the second user input to provide a failure to rescue analysis; and outputting the failure to rescue analysis.

### CROSS-REFERENCE TO RELATED APPLICATIONS

This application claims the benefit of U.S. provisional patent application No. 63/468,651, filed on May 24, 2023, and titled “SYSTEMS AND METHODS FOR PREDICTING AND PREVENTING FAILURE TO RESCUE EVENTS,” the disclosure of which is expressly incorporated herein by reference in its entirety.

### BACKGROUND

A “failure to rescue” is an adverse event following a complication in a healthcare environment. A failure to rescue can occur when a health care provider fails to recognize or diagnose a condition before that condition leads to a complication. Such complications can be dangerous or fatal. Human error can contribute to failures to rescue. Systems and methods that can prevent failure to rescue events can improve healthcare and prevent complications.

### SUMMARY

In some aspects, the techniques described herein relate to a computer-implemented method to prevent a failure to rescue event, the method including: providing a user with a first query; receiving a first user input in response to the first query; providing the user with a second query, wherein the second query is at least partially based on the first user input; receiving a second user input in response to the second query; analyzing at least one of the first user input and the second user input to provide a failure to rescue analysis; and outputting the failure to rescue analysis.

In some aspects, the techniques described herein relate to a computer-implemented method, wherein analyzing at least one of the first user input and the second user input includes performing a comparison of the first user input and the second user input to a branching logic database, and wherein outputting the failure to rescue analysis is based on the comparison.

In some aspects, the techniques described herein relate to a computer-implemented method, wherein the failure to rescue analysis includes a three-point scale.

In some aspects, the techniques described herein relate to a computer-implemented method, wherein the failure to rescue analysis includes a four-point scale.

In some aspects, the techniques described herein relate to a computer-implemented method, wherein the failure to rescue analysis includes a treatment recommendation.

In some aspects, the techniques described herein relate to a computer-implemented method, wherein the failure to rescue analysis includes a predicted diagnosis or a predicted prognosis.

In some aspects, the techniques described herein relate to a computer-implemented method, wherein the failure to rescue analysis includes an analysis of at least one of: sepsis, trauma, cardiac ectopy, geriatric trauma, respiratory distress, respiratory failure, intracranial hemorrhage, stages of sepsis or cardiac arrest.

In some aspects, the techniques described herein relate to a computer-implemented method, wherein the second query is at least partially based on comparing the first query to a branching logic database.

In some aspects, the techniques described herein relate to a computer-implemented method, wherein the second query is at least partially based on the first query.

In some aspects, the techniques described herein relate to a computer-implemented method, wherein the second query is determined by comparing first user input to a database of medical reference values.

In some aspects, the techniques described herein relate to a computer-implemented method, further including generating graphical display data associated with the failure to rescue analysis.

In some aspects, the techniques described herein relate to a computer-implemented method, wherein the graphical display data includes color-coded patient statuses plotted on a clinical warning curve.

In some aspects, the techniques described herein relate to a computer-implemented method for failure to rescue prevention, the method including: receiving an electronic medical record; performing a failure to rescue analysis by comparing the electronic medical record to a branching logic database; and outputting the failure to rescue analysis.

In some aspects, the techniques described herein relate to a computer-implemented method, wherein the failure to rescue analysis includes a three-point scale.

In some aspects, the techniques described herein relate to a computer-implemented method, wherein the failure to rescue analysis includes a four-point scale.

In some aspects, the techniques described herein relate to a computer-implemented method, wherein the failure to rescue analysis includes a treatment recommendation.

In some aspects, the techniques described herein relate to a computer-implemented method, wherein the failure to rescue analysis includes a predicted diagnosis or a predicted prognosis.

In some aspects, the techniques described herein relate to a computer-implemented method, wherein the failure to rescue analysis includes an analysis of at least one of: sepsis, trauma, cardiac ectopy, geriatric trauma, respiratory distress, respiratory failure, intracranial hemorrhage, stages of sepsis or cardiac arrest.

In some aspects, the techniques described herein relate to a computer-implemented method, wherein the branching logic database includes a database of medical reference values.

In some aspects, the techniques described herein relate to a computer-readable medium, wherein execution of the instructions by a processor causes the processor to provide a user with a first query; receive a first user input in response to the first query; provide the user with a second query, wherein the second query is at least partially based on the first user input; receiving a second user input in response to the second query; analyze at least one of the first user input and the second user input to provide a failure to rescue analysis; and output the failure to rescue analysis.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates a flowchart of a computer-implemented method of performing a failure to rescue analysis, according to implementations of the present disclosure.

FIG. 1B illustrates a flowchart of a computer-implemented method of performing a failure to rescue analysis based on electronic medical records, according to implementations of the present disclosure.

FIG. 1C illustrates an example system that can be used to implement the methods of FIGS. 1A and 1B, according to implementations of the present disclosure.

FIG. 2A illustrates an example user interface that can present a user with queries related to a failure to rescue analysis.

FIG. 2B illustrates an example user interface that can present a user with information related to a failure to rescue analysis.

FIG. 3 is an example computing device.

FIG. 4A illustrates an example clinical warning curve directed to neurologic system changes that can be output by a user interface, according to implementations of the present disclosure.

FIG. 4B illustrates an example clinical warning curve directed to respiratory system changes that can be output by a user interface, according to implementations of the present disclosure.

FIG. 4C illustrates an example clinical warning curve directed to cardiovascular system changes that can be output by a user interface, according to implementations of the present disclosure.

FIG. 4D illustrates an example clinical warning curve directed to temperature changes that can be output by a user interface, according to implementations of the present disclosure.

FIG. 4E illustrates an example clinical warning curve directed to lactate levels and mortality that can be output by a user interface, according to implementations of the present disclosure.

FIG. 4F illustrates an example clinical warning curve directed to sepsis-2 changes that can be output by a user interface, according to implementations of the present disclosure.

FIG. 5A illustrates an example interface for providing queries to a user, according to implementations of the present disclosure.

FIG. 5B illustrates an example user interface for outputting a failure to rescue analysis to a user, according to implementations of the present disclosure.

FIG. 5C illustrates an example interface for providing queries to a user, according to implementations of the present disclosure.

FIG. 5D illustrates an example user interface for outputting a failure to rescue analysis to a user, according to implementations of the present disclosure.

FIG. 6A illustrates an example respiratory clinical warning curve that can be output by a user interface, according to implementations of the present disclosure.

FIG. 6B illustrates an example lactate and mortality clinical warning curve that can be output by a user interface, according to implementations of the present disclosure.

FIG. 6C illustrates an example sepsis-2 clinical warning curve that can be output by a user interface, according to implementations of the present disclosure.

FIG. 6D illustrates an example neurologic clinical warning curve that can be output by a user interface, according to implementations of the present disclosure.

FIG. 6E illustrates an example temperature clinical warning curve that can be output by a user interface, according to implementations of the present disclosure.

FIG. 6F illustrates an example cardiac clinical warning curve that can be output by a user interface, according to implementations of the present disclosure.

FIG. 7A illustrates content validity index scores for a study of an example implementation of the present disclosure.

FIG. 7B illustrates inter-rater reliability scores for a study of an example implementation of the present disclosure.

FIG. 7C illustrates perceived usability for a study of an example implementation of the present disclosure.

FIG. 7D illustrates a summary of the system usability scale survey for the study of the example implementations of the present disclosure.

### DETAILED DESCRIPTION

Described herein are systems and related methods for preventing failure to rescue events.

A challenge in clinical practice is systemically identifying patients that are beginning to suffer clinical decline before the patients suffer severe events or complications. For example, a “failure to rescue” is an adverse event following a complication in a healthcare environment. A failure to rescue can occur when a health care provider fails to recognize or diagnose a condition before that condition leads to a complication. Such complications can be dangerous or fatal.

A related challenge to detecting clinical decline is the challenge of instructing, training, and monitoring clinical staff in the clinical environment. For example, less experienced staff (e.g., recently graduated nurses) may lack hands-on experience of failure to rescue events, and may not recognize early signs of clinical decline. This can result in early signs of clinical decline being missed until the clinical decline becomes more obvious (and correspondingly more severe/dangerous). For example, a newer nurse may fail to recognize symptoms of sepsis at the stages of early symptoms (e.g., an elevated heart rate or temperature), and thereby fail to provide sepsis care until the patient is at a later stage of sepsis (e.g., septic shock or severe sepsis). In extreme cases, this can result in death if a patient has severely declined and cannot be rescued (a failure to rescue event).

Implementations of the present disclosure can address these and other challenges in clinical monitoring by providing systems and methods for providing systemic monitoring of patient conditions. The implementations of the present disclosure described herein can detect early stages of clinical decline and/or alert clinical staff (e.g., doctors, nurses, and others) to the clinical decline. For example, implementations of the present disclosure can use a series of queries that are related or linked by branching logic to perform individualized analyses of whether a patient is at risk for a failure to rescue event (e.g., by using a series of queries to assess whether a patient is in an early stage of sepsis). By using branching logic, the queries can be adaptive to different patient conditions and combinations of patient conditions, and can also reliably implement traceable, research-based logical rules.

The systems and methods described herein can be used both directly by staff (e.g., nurses) to assess patients systematically for clinical warning signs, and/or by computer systems (e.g., a patient management system) to automatically detect patient decline based on medical records and monitoring. Additionally, implementations of the present disclosure can provide guidance to clinical staff in performing diagnosis and treatment based on the detected stages of clinical decline that can improve education and training. Accordingly, implementations of the present disclosure include improved systems and methods for preventing failure to rescue events and generally improving diagnosis, treatment, and other clinical practices.

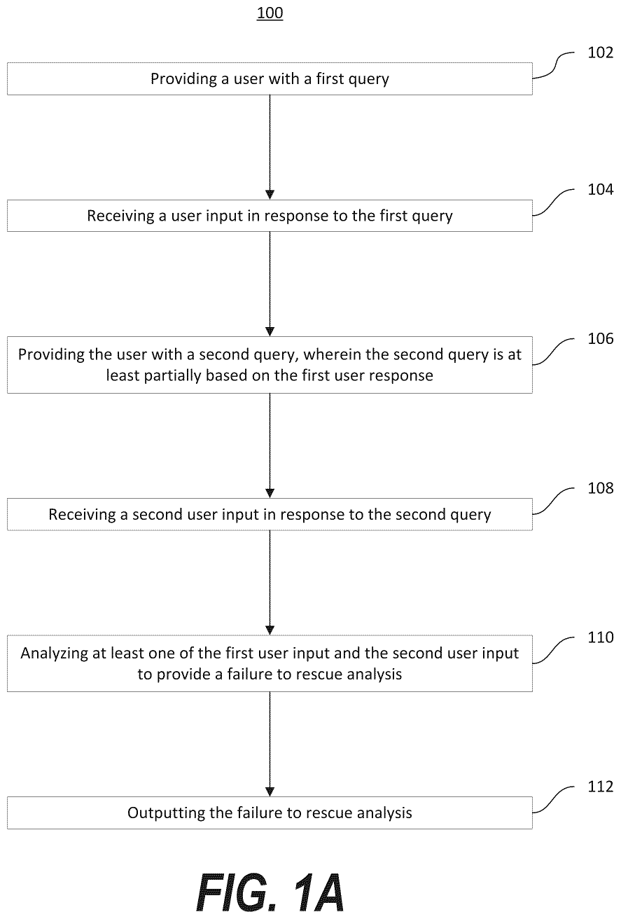

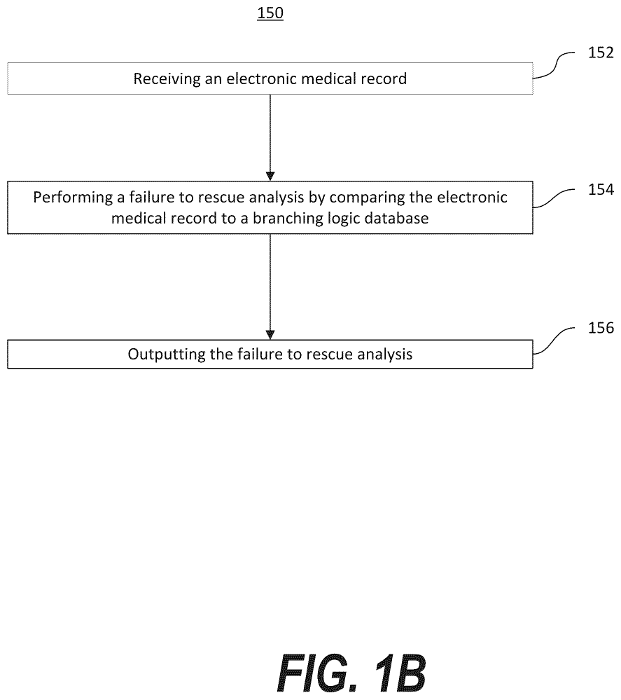

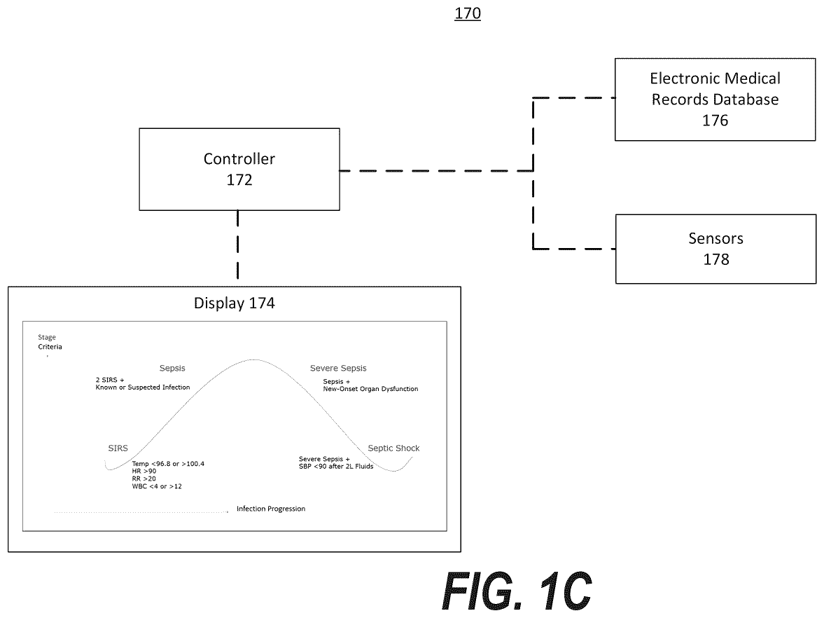

FIG. 1A illustrates a flowchart of an example computer-implemented method 100 for preventing a failure to rescue event.

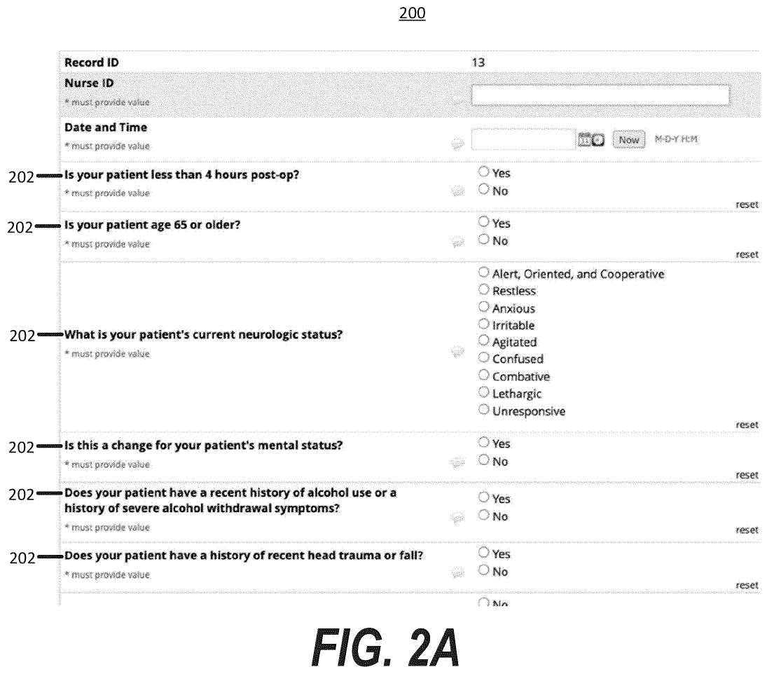

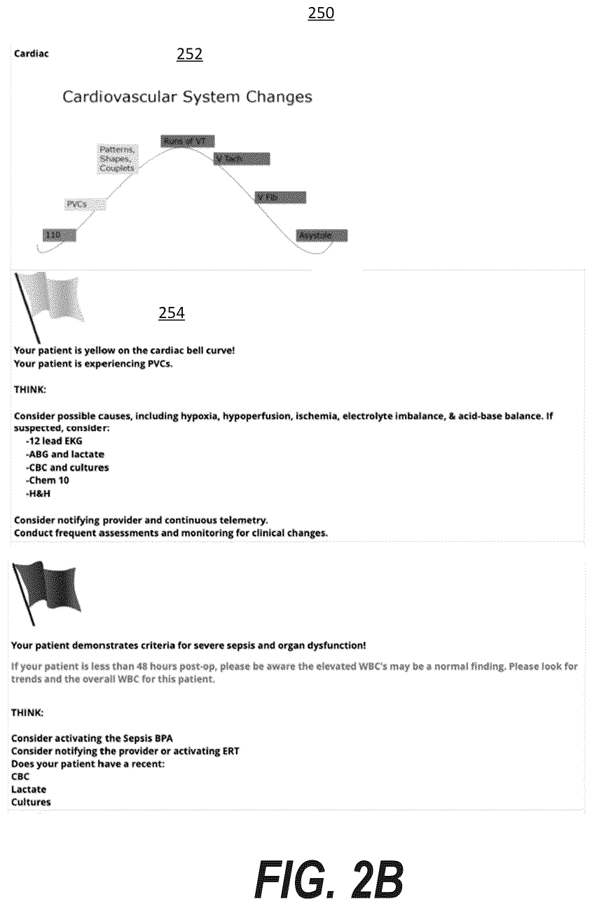

At step 102, the computer-implemented method 100 includes providing a user with a first query. A non-limiting example of an interface 200 for providing a first query is shown in FIG. 2A. The interface 200 can include any number of queries 202, and it should be understood that any pair of the queries can be the first and second query as described herein.

At step 104 the computer-implemented method 100 includes receiving a first user input in response to the first query. Again, the response can be performed using the interface 200 shown in FIG. 2A.

At step 106 the computer-implemented method 100 includes providing the user with a second query, where the second query is at least partially based on the first user input. In some implementations, the second query is at least partially based on the first query. As a non-limiting example, the first query may be a question related to signs of sepsis. If the user input in response to the first query at step 104 suggests that sepsis is present, the second query may be a query configured to confirm the presence of sepsis.

Optionally, the second query can be determined by comparing the first query to a branching logic database. In some implementations, the branching logic database can include a database of medical reference value that the user input in response to the first and/or second query can be compared to.

At step 108, the computer-implemented method 100 further includes receiving a second user input in response to the second query.

At step 110, the computer-implemented method 100 further includes analyzing at least one of the first user input and the second user input to provide a failure to rescue analysis.

In some implementations, analyzing at least one of the first user input and the second user input includes comparing the first user input and the second user input to a branching logic database, and wherein outputting the failure to rescue analysis is based on the comparison.

At step 112 the computer-implemented method 100 further includes outputting the failure to rescue analysis. Optionally, outputting the failure to rescue analysis includes generating graphical display data associated with the failure to rescue analysis. An example user interface 250 for outputting the failure to rescue analysis is shown in FIG. 2B. Optionally, the graphical display data can include color-coded patient statuses plotted on a clinical warning curve 252.

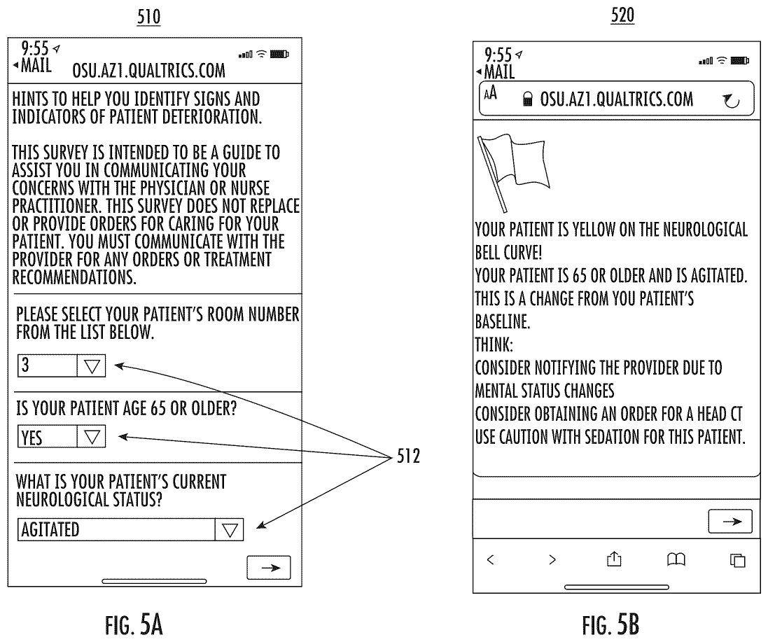

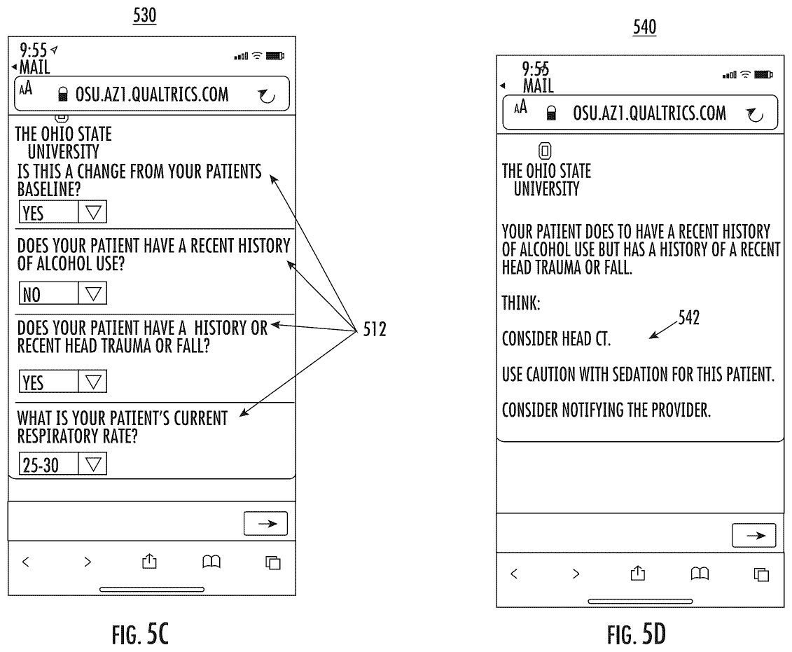

Additional example user interfaces for providing queries and outputting failure to rescue analyses are shown in FIGS. 5A-5C. FIG. 5A illustrates an example interface 510 for providing queries 512 to a user, according to implementations of the present disclosure. FIG. 5C illustrates an additional example interface 530 for providing queries 512 to a user, according to implementations of the present disclosure. It should be understood that the queries shown in FIGS. 5A and 5C are intended only as non-limiting examples of queries.

FIG. 5B illustrates an example user interface 520 for outputting a failure to rescue analysis to a user, according to implementations of the present disclosure. FIG. 5D illustrates an example user interface 540 for outputting a failure to rescue analysis to a user, according to implementations of the present disclosure. As shown in FIG. 5B, the user interface 520 can optionally include a score referring to one or more of the clinical warning curves described herein. Alternatively or additionally, as shown in FIG. 5D, an user interface 540 can include outputting instructions 542 to the user to direct diagnosis and/or treatment of a subject.

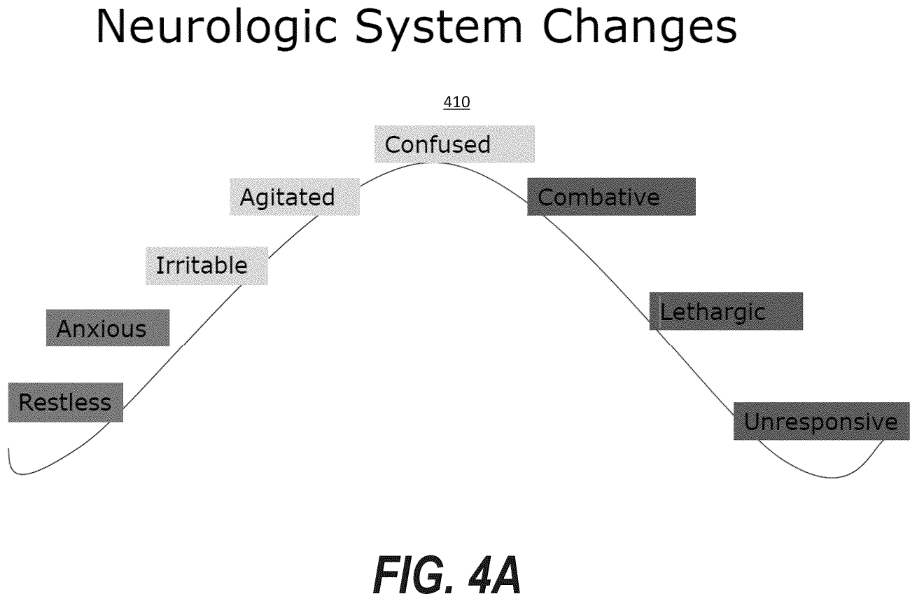

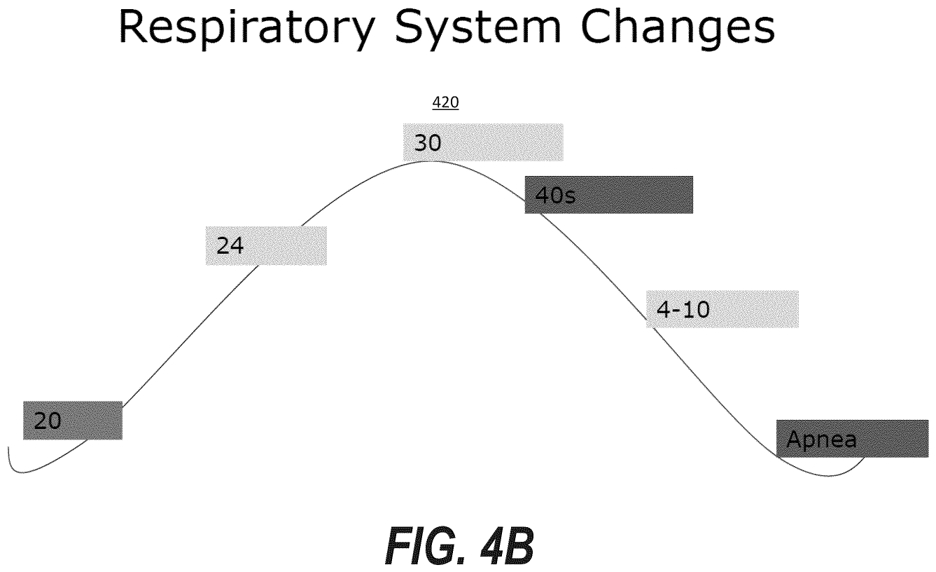

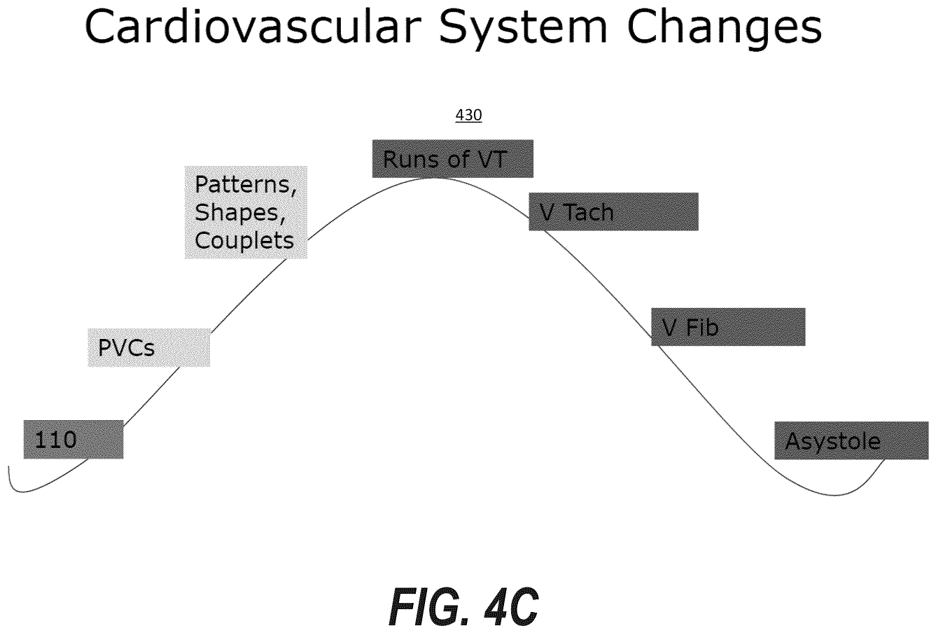

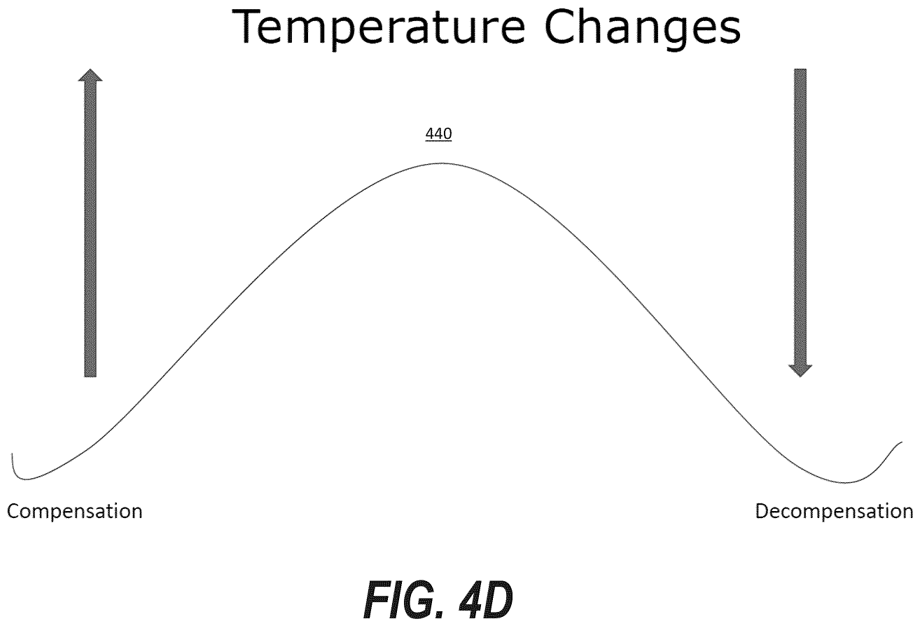

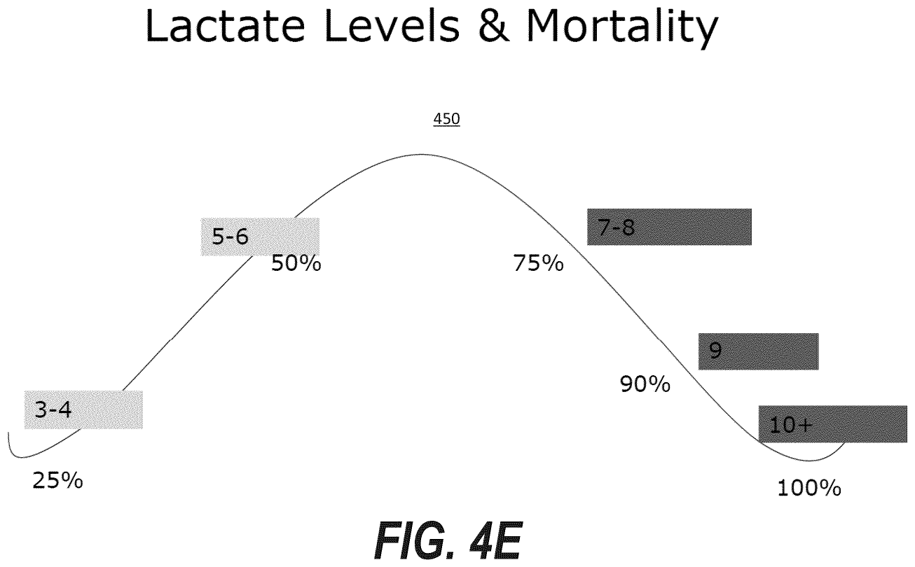

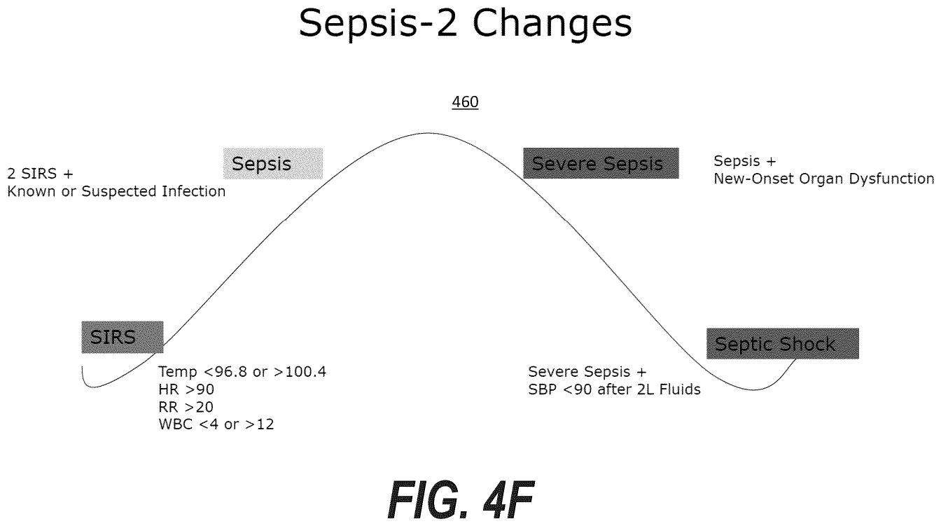

Additional example clinical warning curves that can be used in implementations of the present disclosure are illustrated in FIGS. 4A-4F (e.g., as part of the user interface 250 described in FIG. 2B). FIG. 4A illustrates an example clinical warning curve 410 that can be directed to neurologic system changes, FIG. 4B illustrates an example clinical warning curve 420 that can be directed to respiratory system changes, FIG. 4C illustrates an example clinical warning curve 430 that can be directed to cardiovascular system changes, FIG. 4D illustrates an example clinical warning curve 440 that can be directed to temperature changes, FIG. 4E illustrates an example clinical warning curve 450 that can be directed to lactate levels and mortality, and FIG. 4F illustrates an example clinical 460 warning curve that can be directed to sepsis-2 changes. It should be understood that any number and combination of clinical warning curves can be output, according to different implementations of the present disclosure. It should also be understood that the clinical warning curves illustrated herein are intended only as non-limiting examples, and that additional types of clinical warning curves directed to other conditions are contemplated by the present disclosure.

Optionally, the failure to rescue analysis can include a multi-point scale. In some implementations, as a non-limiting example, the failure to rescue analysis can be a three- or four-point scale. Optionally, different conditions (e.g., sepsis or cardiac arrest) can be assessed on different scales.

Optionally, the failure to rescue analysis includes a treatment recommendation. Non-limiting examples of treatment recommendations 254 and recommendations for further testing are illustrated in FIG. 2B. In some implementations, the failure to rescue analysis can include a predicted diagnosis or a predicted prognosis. Non-limiting examples of diagnoses, prognoses, and analyses that can be performed by implementations of the present disclosure include sepsis, trauma, cardiac ectopy, geriatric trauma, respiratory distress, respiratory failure, intracranial hemorrhage, stages of sepsis or cardiac arrest.

With reference to FIG. 1B, a computer-implemented method 150 for failure to rescue prevention shown. The computer-implemented method 150 includes receiving an electronic medical record at step 152. Optionally the electronic medical record can be received from a specialized hospital computer system.

At step 154, the computer-implemented method includes performing a failure to rescue analysis by comparing the electronic medical record to a branching logic database. Optionally, the branching logic database includes a database of medical reference values.

At step 156, the computer-implemented method further includes outputting the failure to rescue analysis. As described with respect to the computer-implemented method shown in FIG. 1A, the failure to rescue analysis can include a multi-point scale (e.g., three points or four points) and the computer-implemented method can also assess different conditions using different scales. Additionally, as also described with reference to FIG. 1A, the failure to rescue analysis can include a treatment recommendation and/or a predicted diagnosis and/or a predicted prognosis. Non-limiting examples of conditions that can be analyzed by the computer-implemented method 150 include sepsis, trauma, cardiac ectopy, geriatric trauma, respiratory distress, respiratory failure, intracranial hemorrhage, stages of sepsis or cardiac arrest.

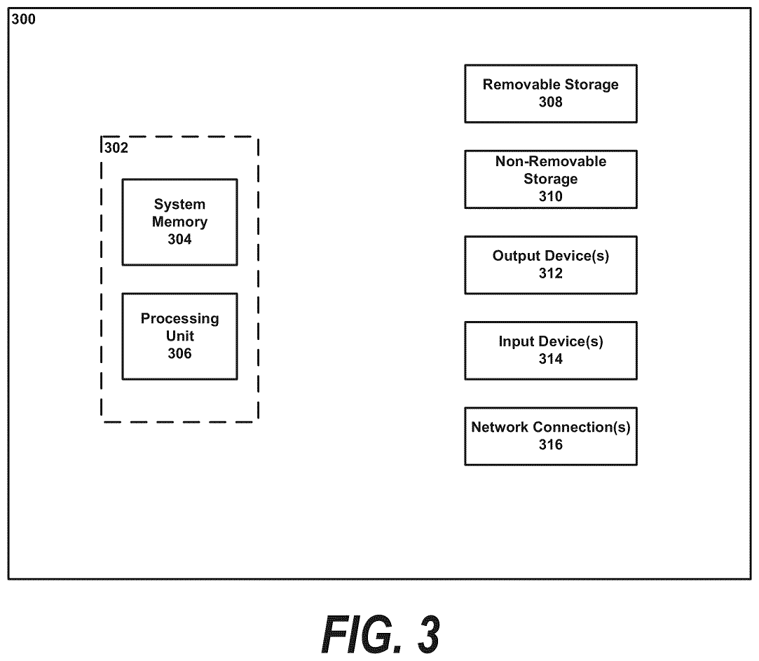

FIG. 1C illustrates an example system 170 that can be used to implement the methods described herein. The system 170 can include a controller 172, display 174, electronic medical records database 176, and sensors 178. The controller 172 can optionally be a mobile computing device or computer terminal located in a hospital or other clinical setting, and the controller 172 can include any or all of the components of the computing device 300 shown in FIG. 3. The controller 172 can be operably connected to the electronic medical records database 176 and/or sensors 178 (for example, using a network).

The present disclosure contemplates that the controller 172 can receive information about the condition of a patient, and use that information to perform a failure to rescue analysis as described herein with reference to FIGS. 1A-1B. For example, the controller 172 can receive information about the condition of a subject from the electronic medical records database 176 and/or sensors 178, and use that information to perform a failure to rescue analysis. Optionally, the controller 172 can be configured to provide a user with queries that can be used as part of the failure to rescue analysis, and/or to output a failure to rescue analysis based on the answer to those queries. For example, the controller can be operably coupled to a display 174 (e.g., part of a mobile computing device or hospital computer terminal) to output failure to rescue analyses and/or clinical warning curves. Alternatively or additionally, the controller 172 can be configured to output diagnosis and/or treatment information and/or instructions. Non-limiting examples of interfaces that can be displayed using the display 174 are described with reference to FIGS. 2A-2B, and 4A-7D herein.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° F. or is at ambient temperature, and pressure is at or near atmospheric.

### Example 1

Example implementations of the present disclosure were studied. The example implementation includes a program referred to herein as Treatment and Help Implementing Nursing Knowledge (“THINK”).

Failure to rescue (FTR) is broadly defined as an adverse event following the development of a complication that was unrecognized by providers. FTR is a national concern. Garvey (2015) created the Clinical Warning Curve Models that depict the progression of clinical changes that occur prior to cardiac arrest in the neurological, pulmonary, and cardiovascular systems. A retrospective chart review of 60 cardiac arrest charts was conducted to identify clinical changes seen within 48-72 hours prior to cardiac arrest. Combining the results with evidence-based sources, three Clinical Warning Curve diagrams were created that depict the changes that occur prior to cardiac arrest. Three more Clinical Warning Curves (also referred to herein as “Curves”) depict body temperature changes prior to cardiac arrest, the relationship between lactate levels and mortality, and the stages of sepsis according to the Sepsis-2 classification. However, even with predictable changes, recognition remains a problem (Considine et al., 2020; Jones & Johnstone, 2017; Sterner et al., 2019; Wong et al., 2017).

A leading cause of FTR is unrecognized sepsis. O'Shaughnessy et al. (2017) report that up to one-quarter of medical/surgical patients develop sepsis, but less than 40% of nurses recognize the symptoms. Similarly, the Surviving Sepsis Campaign reported a 44% mortality for patients with severe sepsis on medical/surgical units that was caused by delayed recognition, delayed treatment, and providers attributing symptoms to the underlying diagnosis (Schorr, 2016).

Studies of FTR in the trauma population reveal that due to age and comorbidities, geriatric trauma patients are more likely to experience an adverse event and FTR; fortunately, the risk of FTR is strongly-linked to modifiable hospital characteristics and processes (Earl-Royal, 2016; Kuo et al., 2017; Roussas et al., 2020; Scantling et al., 2020; Sharoky et al., 2019). Similarly, geriatric patients demonstrate higher mortality rates following trauma compared to younger patients, and that mortality rates increase hierarchically as FTR rates increase (Barmparas et al., 2018).

The THINK was created to proactively stop a failure to rescue (FTR) event. The THINK can be completed in less than two minutes and is compatible with any web browser. The nurse can complete the THINK at the start of shift and with any changes in assessment. The THINK can use radio-dial formatted answers to rate the patient's status with vital signs, physical assessment, and laboratory results and places the patient onto the Garvey Clinical Warning Curves. Using a mapping algorithm, the THINK can provide recommendations and things to “think” about regarding the patient's care.

Based on the answers, the THINK provides an output predicting failure to rescue events and potential treatments.

The THINK Survey, and other data can be housed in a secure server (e.g., a HIPAA-compliant REDCap platform).

THINK can assist all nurses with recognition and management of clinical deterioration, which will decrease the number of FTR events and associated complications and mortality. THINK can be used by any hospital, even internationally.

Optionally, THINK can be configured to operate with hospital computer systems such as EPIC to auto-populate fields in the THINK. THINK can then be integrated into hospital computer systems, including any system that stores electronic medical records.

While implementations described herein are described with reference to nurses and nursing, THINK can optionally be used by physicians, interns, residents, and/or any other healthcare workers. Optionally, THINK can be configured so that nurses and physicians are presented with can use the same system to implement THINK and communicate regarding signs and symptoms of clinical decline to prevent failure to rescue events. Optionally, the same system can present different information and request different inputs from the physicians and nurses.

### Example 2

An example implementation of the present disclosure was configured for use in clinical practice by nurses, and a study was performed of the example implementation. New graduate nurses can have difficulty recognizing and managing the early signs of clinical decline, resulting in a Failure to Rescue (FTR) event and preventable patient death. To address this gap, a systems and methods including Clinical Warning Curves were developed. The example study included Clinical Warning Curve models to depict the progression of clinical changes that occur before cardiac arrest in six body systems.

The study was a cross-sectional, observational, validation survey design. The purpose of this study was to establish the content validity, reliability, and usability of the example Clinical Warning Curves among healthcare experts and new-graduate nurses. Content experts used the content validity index (CVI) to evaluate the Curves. All but the temperature Curve were rated as “acceptable” (mean CVI>2.5) for relevance, clarity, and ambiguity. The new graduate nurses who reviewed the case studies and placed patients onto the Clinical Warning Curves did so similarly, generating high intraclass correlation (ICC) scores. The usability survey components measured the perceptions of acceptability, appropriateness, and feasibility for the use of the six Clinical Warning Curves in practice settings. All components of the Curves had an average score of 4.0 or greater except for the level of complexity which scored 3.88.

The results of the study show that the example systems and methods using Clinical Warning Curves are valid and reliable tools that aid new graduate nurses in recognizing subtle signs of clinical decline. Because timely recognition and communication of clinical decline are key to preventing FTR events and avoiding patient deaths, the example implementation including Clinical Warning Curves can improve treatment by new nurses in a clinical setting.

As described in the study, Failure to rescue (FTR) is broadly defined as an adverse event following the development of a complication that was unrecognized by providers, is a national concern (Barmparas et al., 2016). A FTR event may culminate in cardiac arrest, with only 50% of patients being successfully resuscitated (Zegrean, et al., 2023). Many tools have been developed to aid healthcare clinicians in recognizing early signs of patient deterioration, such as the Modified Early Warning Score (MEWS) which quantifies clinical decline using a numerical scale (Zegrean, et al., 2023). Epic, a leading electronic medical record (EMR) developer, created the sepsis predictive analytic tool that searches the EMR looking for clinical variables and other signs of sepsis. If the signs are identified, the tool sends an alert to notify providers of the possibility of sepsis (Cull, et al., 2023). However, these tools are geared toward the experienced clinician and are often difficult for new graduate nurses to integrate into their routine assessment and practices. To address this gap, the example implementation of the present disclosure includes a series of Clinical Warning Curves as an instructional tool for new graduate nurses (2015). Preliminary use of the tools in clinical settings has been positive for nurse educators, new nurses, and precepting nurses. Therefore, a purpose of this study was to evaluate the validity, reliability, and end-user perceptions of the usability of the example Clinical Warning Curves.

Failure to rescue (FTR) remains an issue, particularly with new-graduate nurses. Patients can display subtle signs of clinical decline up to 48 hours before a cardiac arrest, and new-graduate nurses tend to have difficulty recognizing these signs and managing the causative conditions (Zegrean, et al., 2023). Several things contribute to this issue, including a lack of previous experiences with declining patients, overwhelming workloads, and inadequate staffing (Chua, et al., 2019). The early signs of clinical decline are very subtle and easily attributed to benign causes, such as mental status changes being attributed to hospital delirium. Other leading causes of FTR events are ineffective communication and the medical hierarchy that places nurses as subservient to physicians (Chua et al., 2019). New-graduate nurses often lack confidence in their assessment skills, and when combined with the subtle presentation of early clinical decline, ineffective communication, and the medical hierarchy, they fail to recognize the early signs of clinical decline or effectively communicate their concerns to the provider (Chua, et al., 2020). As a result, the FTR event progresses into a cardiac arrest, from which 50% of patients do not recover (Zegrean, et al., 2023).

Subtle mental status changes, including anxiety and restlessness, are the first indicators of clinical decline that often result from hypoperfusion or hypoxia (Dennison, 2018). The subtlety of these indicators can lead to FTR events, when due to lack of strong assessment skills and ineffective communication, the nurse only shares with the provider that the patient is restless and anxious. This results in an improper diagnosis of hospital delirium and the administration of sedatives that further decrease the patient's respiratory drive, worsen hypoxia, and ultimately result in cardiac arrest. The second indicator of clinical decline is an increased respiratory rate. A respiratory rate of 20 breaths per minute is one of the earliest signs of deterioration and is also a criterion of systemic inflammatory response syndrome (SIRS), 2015; Levy, et al., 2018). Through interactions with nurses in the monthly FTR Prevention course, it became evident that many nurses do not take the time to count the respiratory rates of their patients. As a result, the second early sign of clinical decline is often missed and contributes to a FTR event.

Existing tools include significant drawbacks, for example, they may automatically populate without any prompting by the nurse, and may generate more alarms in the already alarm-overloaded clinical setting. Because new-graduate nurses struggle with overwhelming workloads, inadequate staffing, and alarm fatigue, they become task-focused and unable to step back to see the bigger picture of their patient's status. Although the MEWS accurately predicts clinical decline, new graduate nurses are often unsure of what each number means and fail to act on the information. Because of these struggles and the nurse not actively soliciting notifications from these tools, the nurse does not heed the warning and quickly dismisses the notifications, leading to FTR events (Cull, et al., 2023).

Nurses are in an optimal position to recognize early signs of clinical decline; however, they need education and interactive tools to foster that recognition (Wong, et al., 2017; Zegrean, et al., 2023). The study included the curves described in Example 1 herein, and three more Curves for temperature changes, lactate levels and mortality, and sepsis-2 stages. The study included a literature review to ensure that the stages listed on each Curve remained accurate and current. The literature review encompassed multiple databases, including EBSCO and CINAHL, and leading books in the field. Keywords used to search the literature included failure to rescue, sepsis, lactate levels, hypoxic changes, neurological changes, respiratory distress, new graduate nurses, and clinical decline.

### Design of Clinical Warning Curves

The present disclosure contemplates using a variety of Clinical Warning Curves, which can be updated to use different standards and research parameters.

For example, the original sepsis-2 Clinical Warning Curve uses the four stages of sepsis as defined by Rhodes, et al. (2017), including systemic inflammatory response syndrome (SIRS), sepsis, severe sepsis, and septic shock. Since then, a new definition of sepsis and septic shock appeared, known as sepsis-3. In short, sepsis-3 removed the category of SIRS, operationalized the classification of severe sepsis by implementing the Sequential Organ Failure Assessment (SOFA), and altered the definition of septic shock (Shankar-Hari, et al., 2017). The literature review reflected continued support of the sepsis-2 criteria, finding that more hospitalized patients met the sepsis-2 criteria, and the SIRS criteria were more sensitive to the prediction of in-hospital mortality (Radigan, 2018; Shankar-Hari et al., 2017; Vermassen et al., 2021).

The content of the temperature Curve, the respiratory Curve, and the lactate levels and mortality rates Curve remains accurate (Dennison, 2018; Levy, et al., 2018; Rhodes, et al., 2017). Recent literature supports adding irregularity or new-onset atrial fibrillation as early changes to the cardiac Curve and validates new-onset tachycardia as an early warning indicator of clinical decline (Dennison, 2018; Huff, 2016). Finally, Dennison (2018) continues to support the progression of mental status changes on the neurologic Curve. To expand upon preliminary feedback of the Curves, the team sought to formally establish content validity, as well as evaluate reliability and usability among the target population of new graduate nurses. Results reflected satisfactory content validity, reliability, and usability of the Clinical Warning Curves. It should be understood that these data sources and curves are intended only as non-limiting examples, and the systems and methods described herein are intended to be used with any combination of clinical data, research data, and/or standards.

### Study Design, Aims, and Setting

The study was a cross-sectional, observational, validation survey design. The study established the content validity, reliability, and usability of the Clinical Warning Curves among expert healthcare clinicians and new-graduate nurses. Specific aims of the study included: 1. Establish content validity of the six Clinical Warning Curves among a panel of critical care nursing experts, and 2. Evaluate the inter-rater reliability and usability of the six Clinical Warning Curves among a cohort of new graduate nurses within a large, academic medical center in the midwestern United States.

### Study Sample

Purposive sampling was used to identify the content experts for Aim 1.

Individuals were selected based on their reputations as national leaders in critical care nursing and sepsis, as well as years of experience. These individuals received an email invitation describing the purpose and procedures of the study. A survey link was included in the email for them to voluntarily complete anonymously. Completion of the survey implied their consent to the study procedures.

Convenience sampling was used to identify new graduates who were willing to voluntarily use the tools in case scenarios to establish reliability and to provide their perceptions on the usability of the tools for their clinical practice. New graduate nurses who had completed their baccalaureate nursing education and successfully passed the required state licensing examination to practice as a registered nurse within the prior 12 months were identified from the nurse residency program within the academic medical center that served as the study site. The Clinical Warning Curves are routinely used as instructional tools with new graduates in the nurse residency program, so these new graduate nurses were contacted by email by a representative of the nurse residency program and asked to voluntarily provide feedback on the use of the tools. The new graduate nurses were provided information in the email on the purpose and procedures of the study, and their voluntary completion of the online survey indicated their willingness to participate. The survey link was provided in the email, and all responses were anonymous.

### Study Procedures

To establish the content validity of the Clinical Warning Curves, the identified content experts were asked via email invitation to evaluate each Curve using the procedures outlined by Lynn (1986). These procedures included each expert rating each Curve across the domains of relevance, clarity, simplicity, and ambiguity using a 1-4 Likert scale where 1=not relevant (and not clear, not simple, and doubtful) and 4=very relevant (and very clear, very simple, and meaning is clear). Responses were then aggregated across all raters for each domain for each Curve and summarized to establish a content validity index (CVI). Experts were asked to complete the CVI one time for each Curve.

To evaluate the reliability and end-user perceptions of the Curves, new graduate nurses voluntarily completed a series of case studies and usability questions via email invitation. There were three case studies for the neurological, respiratory, cardiac, and sepsis-2 Curves and two case studies for the temperature and lactate level and mortality Curves. Each new graduate was asked to read the case studies and indicate how they would rate the patient on each of the Clinical Warning Curves. Respondents were also asked to indicate their degree of confidence for each rating provided on a 1-5 Likert scale, where 1=not at all confident and 5=very confident. Scores were then summed across raters for each scenario, and an intraclass correlation coefficient was calculated to determine the degree of agreement among raters for each scenario.

To evaluate new graduate nurses' perceptions of the usability of the tool, a series of questions were included as part of the emailed survey link. Questions were based on Roger's Diffusion of Innovations theory (Rogers, 2003) and established attributes known to influence perceptions of routine use. These included end-user perceptions of the relative advantage, compatibility, adaptability, and complexity of the Curves for use in practice settings. (Rogers, 2003). Additional questions on perceptions of usability included evaluation of the acceptability, appropriateness, and feasibility using survey items from the field of implementation science (Weiner, et al., 2017). Each construct was evaluated using a 5 item Likert scale where 1=completely disagree and 5=completely agree.

### Study Results

The content validity survey was sent to 5 nurse experts. Of these, all 5 (100%) completed the content validity survey. All experts were nurses who specialize in critical care (n=5, 100%), and together, they have an average 33 years of critical care experience (mean=33, range 23-49). Furthermore, 60% (n=3) of the nurse experts are licensed as advanced practice registered nurses (APRN) and 60% (n=3) have their Critical Care Registered Nurse (CCRN) certification.

The reliability and usability surveys were sent to 59 new graduate nurse residents. Among the new graduate nurse sample, a total of 9 individuals completed the reliability and usability surveys, reflecting a 15% response rate. Within this sample, respondents reported an average of 1.5 years in practice (range 0.5-6 years). One respondent had not previously worked in an acute care setting and was thus categorized as a new graduate nurse, despite having licensure for more than 12 months. Within this sample, n=1 (11%) reported having their CCRN certification and all (n=9, 100%) reported having a baccalaureate degree for entry into practice. Of the sample, the majority (n=5, 63%) practice in critical care. The following sections explain the results of the content validity findings from the nursing experts (Aim 1) and the usability and inter-rater reliability results from the nursing end-users (Aim 2).

### Aim 1: Content Validity of the Six Clinical Warning Curves

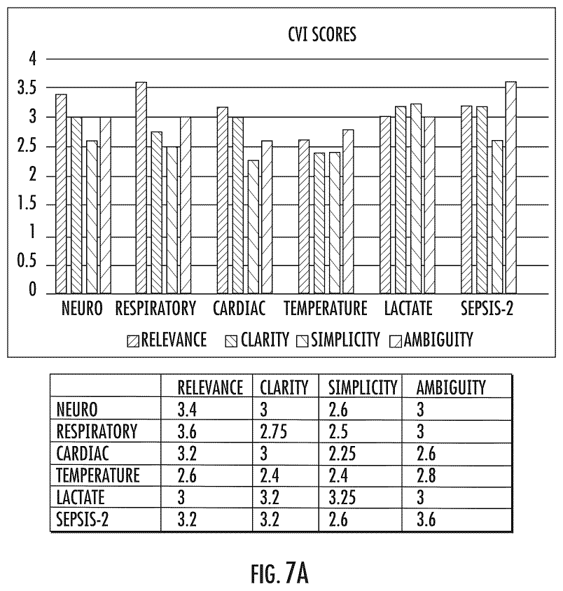

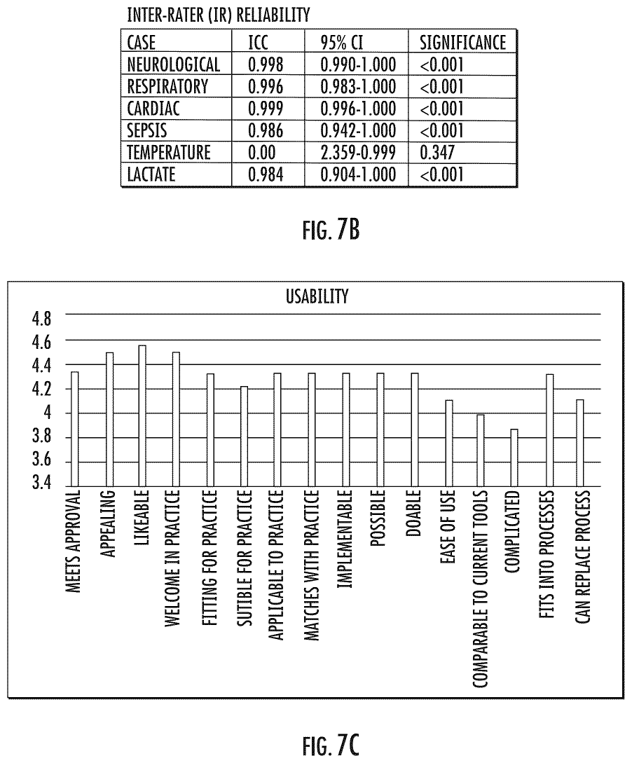

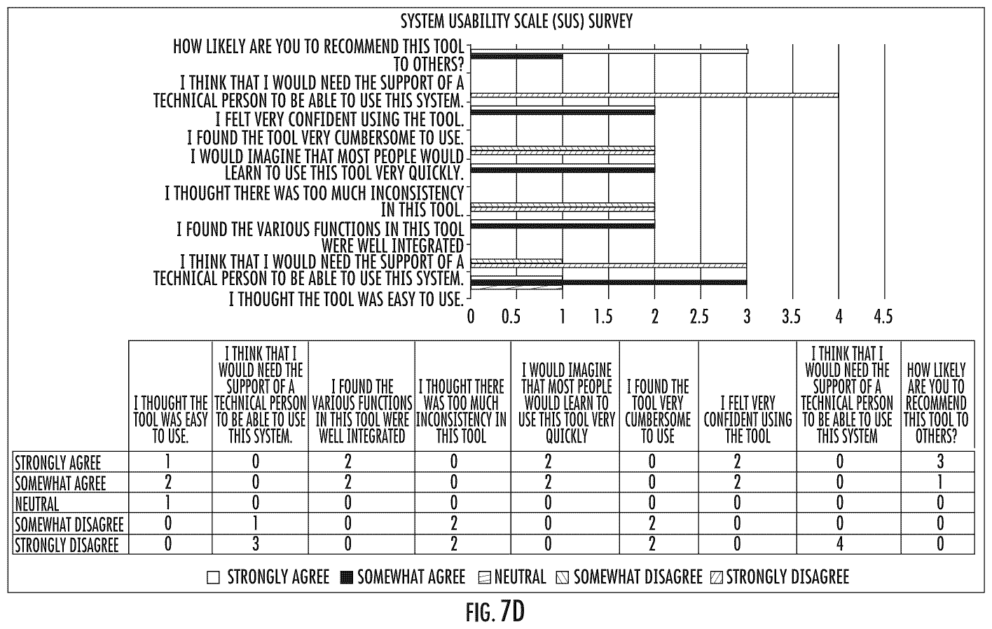

FIG. 7A, Content Validity Index (CVI) Scores, illustrates the average CVI scores aggregated across experts. As displayed in FIG. 7A, all experts rated the Curves acceptable (mean CVI>2.5) for relevance, clarity, and ambiguity for all Curves, except the temperature Curve. The raters indicated the lowest CVI scores for the simplicity of the Curves, except for the lactate Curve. Among the different types of Curves, raters scored the sepsis Curve highest overall for content validity, followed by the lactate Curve, neurologic Curve, respiratory Curve, cardiac Curve, and temperature Curve. The critical care nursing experts also had the opportunity to give written feedback on what remained unclear about the Curves and suggestions for improvement. The most common comments included requests to name the x and y axes to clarify that the stages are a deviation from baseline and that the Curves are excellent for identifying patients who are declining.

### Aim 2: Inter-rater Reliability and Usability of the Six Clinical Warning Curves

Among the new graduate nurses who reviewed the case studies and reported on their ratings for each scenario using the Clinical Warning Curves, many scored scenarios similarly. In 13 of the 15 case studies, n=7 (78%) most respondents placed the patient correctly on each Curve. In the temperature Curve case studies, n=6 (67%) of respondents correctly placed the patient on the Curve. In the first sepsis case study, only n=4 (44%) of respondents correctly placed the patient on the Curve. Respondents most frequently indicated that they were “somewhat confident” in their responses and showed the most confidence in answering the cardiac Curve case studies. Five of the Curves had high intraclass correlation (ICC) scores. The cardiac Curve scored highest at 0.999 ICC (p=0.001) and the temperature Curve scored the lowest at 0.00 ICC (p=0.347) as shown in FIG. 7B, Inter-rater [IR] Reliability.

The usability survey components for the new graduate nurses aligned with outcomes from the field of implementation science regarding perceptions of acceptability, appropriateness, and feasibility for the use of the six Clinical Warning Curves in practice settings. In total, sixteen components of usability were measured on a 5-point scale where 1=completely disagree and 5=completely agree as shown in FIG. 7C, Perceived Usability. In general, the closer to 5 a component scored, the greater its usability. However, for complexity, a lower score is desired because this would indicate that participants believe the tool is not complicated to use. When applied to the six Clinical Warning Curves, all components of usability had an average score of 4.0 or greater except for the level of complexity which scored 3.88. Like the critical care nursing experts, new graduate nurses had the opportunity to give written feedback on the Curves. Comments included that the six Clinical Warning Curves are easy to look at and determine where your patient ranks, and several participants requested to make the Curves into a badge reel card. FIG. 7D illustrates a summary of the system usability scale survey for the study of the example implementations of the present disclosure.

### Example Alternative Implementations of the Present Disclosure

The study included revising the Clinical Warning Curves to create additional example Clinical Warning Curves.

Findings from the study indicate that the Clinical Warning Curves are a valid and reliable tool that can help new graduate nurses identify patients in clinical decline and prevent FTR events. New graduate nurses also indicated high usability of the Curves. These findings support the use of this tool among new graduate nurses to support their transition to practice and care of complex patients. Providing realistic scenarios and tools for new graduate nurses to enhance their recognition of the signs of early clinical decline may promote increased awareness of these signs, prompt early management of the precipitating condition, and prevent FTR events.

Because timely recognition and communication of clinical decline are key to preventing FTR events and avoidable patient deaths, it would be beneficial to provide the Clinical Warning Curves as a bedside resource for new graduate nurses during their orientation to the unit or within a nurse residency program. Nurse residency programs were created to promote practice readiness (Trepanier et al., 2020). The FTR Prevention class was added to the academic medical center's (study site) nurse residency program in 2016. During this class, nurse residents were given a pocket card that displayed the six Clinical Warning Curves. Based on nurse residents' comments and this study's survey feedback, it would also be feasible to make the Curves into a badge reel card or “badge buddy” to be placed behind the nurse's work ID, allowing constant access to the resource.

Regardless of the distribution method, supplemental education can be provided with the Curves. Traditionally, new graduate nurses are introduced to the Clinical Warning Curves through a four-hour FTR Prevention class, approximately six months into their nurse residency program. During the class, the Clinical Warning Curves are explained in detail, and the nurse residents apply the information through a variety of interactive case studies and a high-fidelity simulation exercise. Based on evaluations received after the class, most nurse residents found the class beneficial for helping them identify early signs of clinical decline and prevent FTR events.

This study supports prior findings in the literature citing new graduate nurses' challenges with recognition of early signs of clinical decline and management of the precipitating conditions (Chua et al., 2019; Herron, 2017; Sterner et al., 2019). Research supports the need to provide new graduate nurses with education and tools to enhance their assessment and management skills (Chua et al., 2019; Sterner et al., 2019). This study revealed satisfactory validity of the Clinical Warning Curves, and the study sample of new graduate nurses reported good reliability and usability of the Curves. The Clinical Warning Curves would be a valuable tool for new graduate nurses to mitigate FTR events.

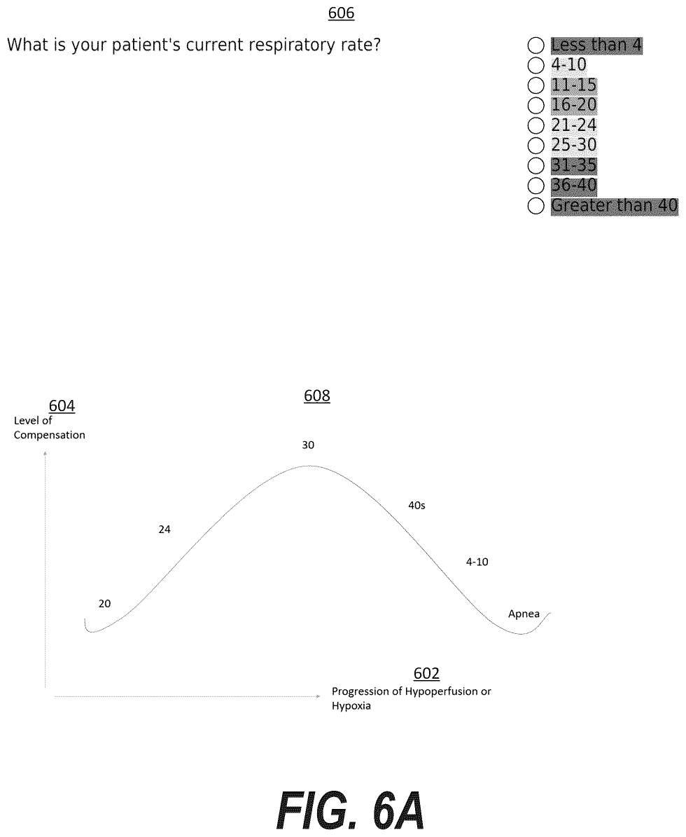

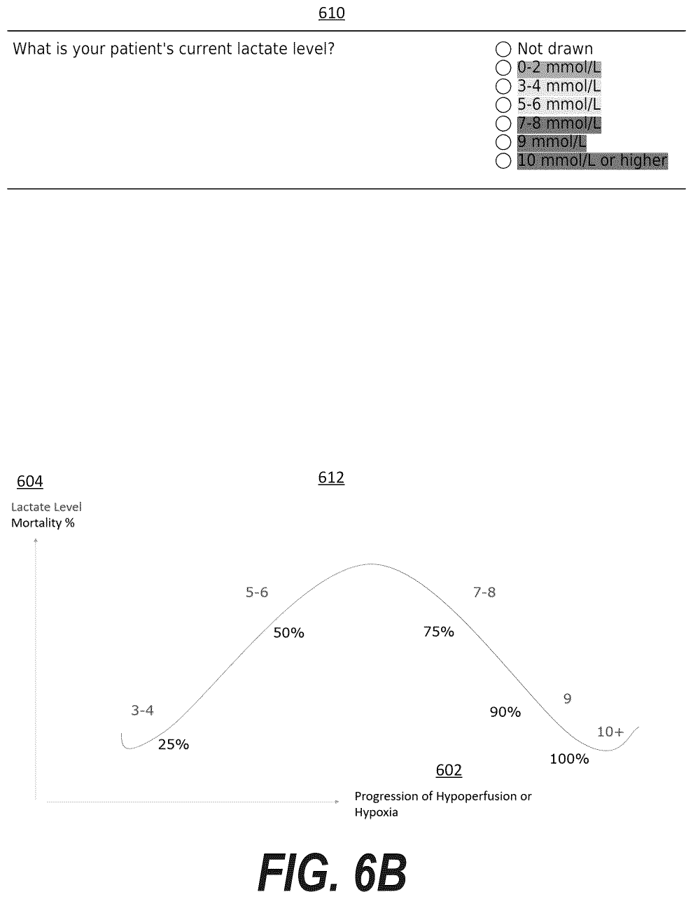

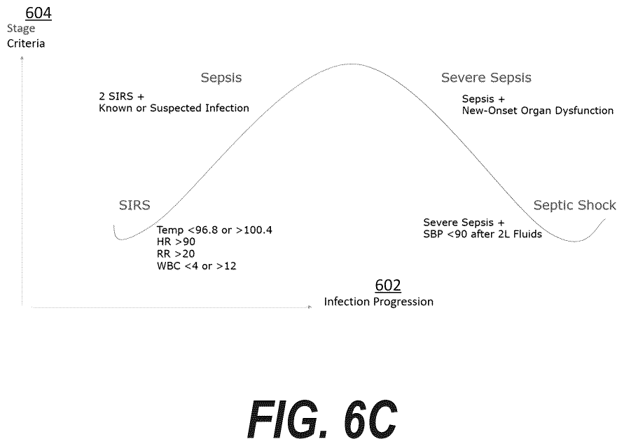

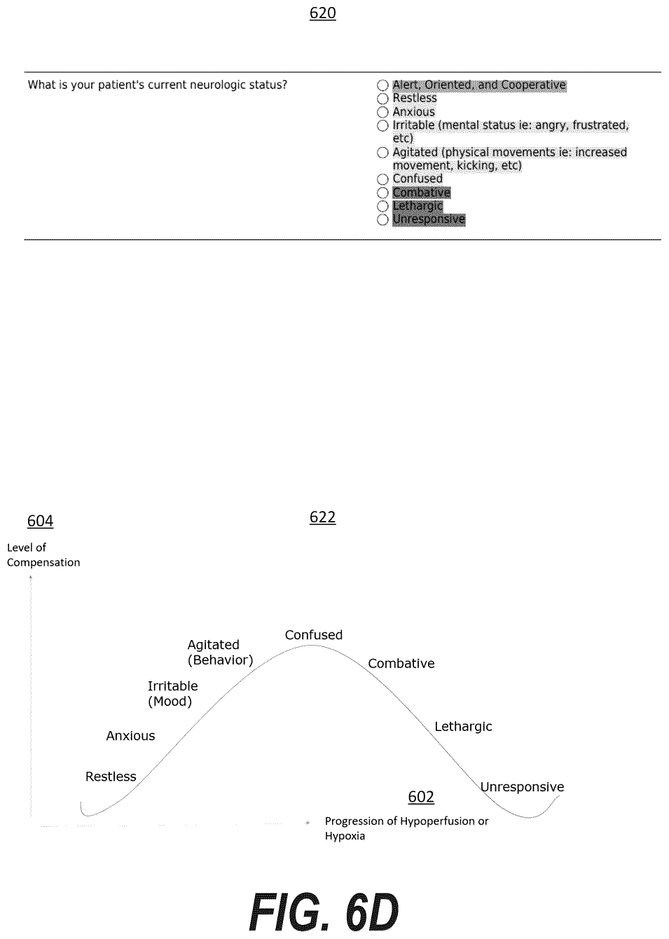

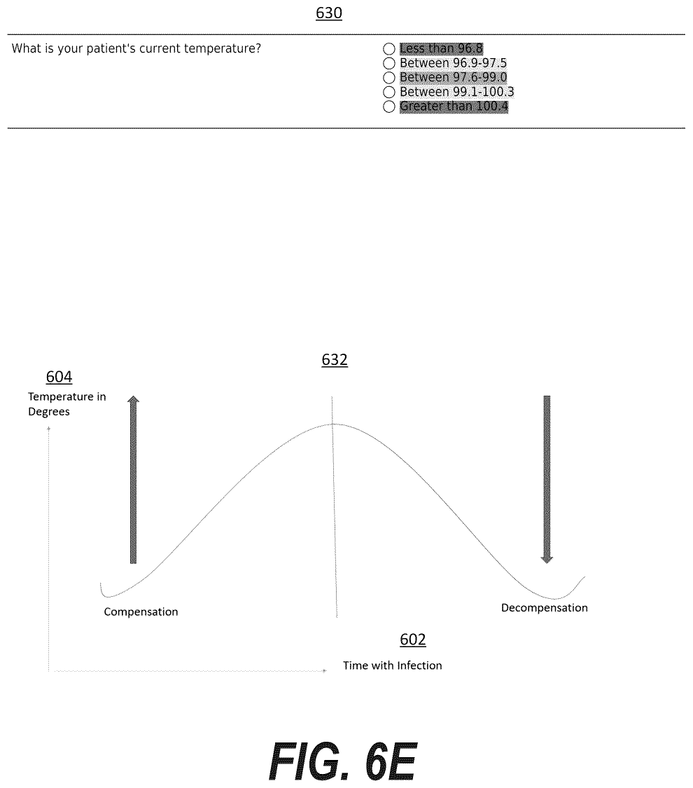

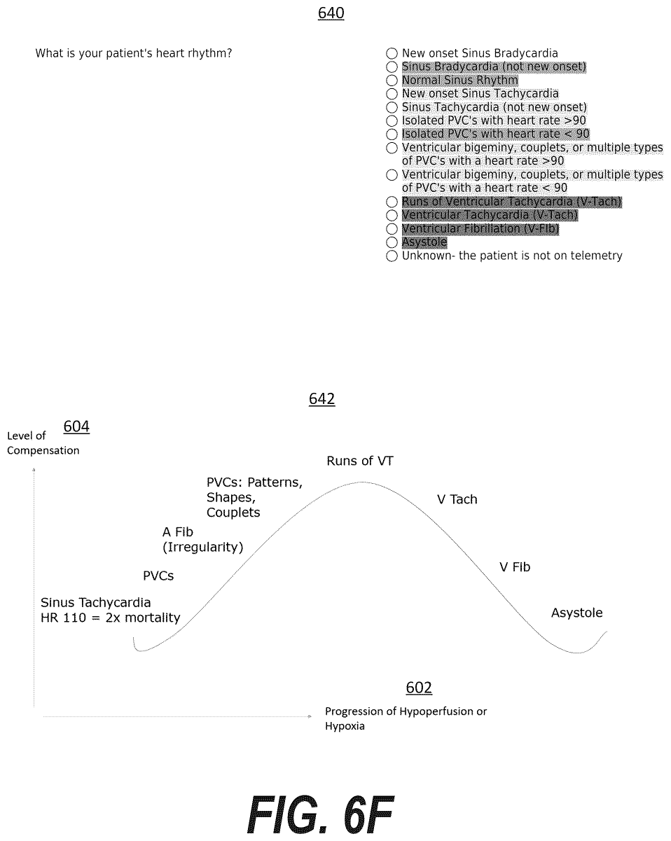

Following the literature review and results of this study, the x-axis 602 and y-axis 604 were labeled on the example Clinical Warning Curves shown in FIGS. 6A-6F. One stage of the cardiac Curve was added based on the experts' feedback. No comments or suggestions were received regarding the respiratory Curve or the lactate and mortality Curve, and the literature review supported the existing stages of these Curves; therefore, no changes were made as shown in FIG. 6A, Respiratory Clinical Warning Curve and FIG. 6B, Lactate and Mortality Clinical Warning Curve. Finally, the stages to the sepsis-2 Curve were verified in the literature and were without change as shown in FIG. 6C, Sepsis-2 Clinical Warning Curve.

The present disclosure contemplates that the x-axis 602 and y-axis 604 can correspond to any clinical parameters with any units. For example, in FIG. 6B, the x-axis 602 can be labelled progression of hypoperfusion or hypoxia and the y-axis 604 can be labelled level of compensation. Example x-axis 602 and y-axis 604 labels were added to the neurological, respiratory, and cardiac Curves based on participants' feedback. The notations of irritable (mood) and agitation (behavior) were added to the neurological curve illustrated in FIG. 6D. On the sepsis-2 Curve, the x-axis label (infection progression) and the y-axis (stage/criteria) were added. On the temperature Curve, the x-axis label (time with infection) and the y-axis label (temperature in degrees Fahrenheit) were added following validation of the experts' comments (Dennison, 2018; Levy, et al., 2018) as shown in FIG. 6E, Temperature Clinical Warning Curve. Following this study, the team also verified the experts' suggestions on the cardiac Curve (Dennison, 2018; Huff, 2016) and altered the Curve to show the addition of atrial fibrillation as shown in FIG. 6F, Cardiac Clinical Warning Curve.

A patient can be placed on the curves using one or more questions provided to a healthcare worker and/or patient. The questions and answers can be classified using color codes representing the likelihood of a failure to rescue event. A green classification corresponds to a low risk, a yellow classification corresponds to an intermediate risk, and a red classification corresponds to a high risk of a failure to rescue event

For example, FIG. 6A illustrates an example respiratory rate question 606 related to. 11-15 and 16-20 are classified “green,” 4-10, 21-24, 25-30 are classified “yellow” and “less than 4” “31-35” “36-40” and greater than 40 are classified “red.” The answer to the respiratory rate question 606 can be plotted on the respiratory rate curve 608.

FIG. 6B illustrates an example lactate level question 610 where 0-2 mmol/L are classified as “green,” 3-4 and 5-6 mmol/L are classified as “yellow,” and 7-8, 9, and 10 mmol/L are classified as “red.” The results can be plotted on the lactate level curve 612.

FIG. 6D illustrates an example neurologic status question 620 where “alert, oriented and cooperative” is classified as “green; restless, anxious, irritable, agitated, and confused are classified as “yellow;” and combative, lethargic, and unresponsive are classified as “red.” The results can be plotted on the neurologic status curve 622.

FIG. 6E illustrates an example temperature status question 630, where “less than 69.8” and “greater than 100.4” are classified as “red;” between 96.9-97.5 and 99.1-100.3 are classified as “yellow” and “97.6-99” are classified as “green.” The results can be plotted on the temperature curve 632.

FIG. 6F illustrates an example cardiac curve 642 with corresponding cardiac status question 640. The following responses are classified “green:” Sinus Bradycardia (not new onset); Normal Sinus Rhythm; Isolated PVC's with heart rate<90 The following responses are classified “yellow:” New onset Sinus Tachycardia, Sinus Tachycardia (not new onset), Isolated PVC's with heart rate>90, Ventricular bigeminy, couplets or multiple types of PVC's with a heart rate>90, and Ventricular bigeminy, couplets, or multiple types of PVC's with a heart rate<90. The following responses are classified “red:” Runs of Ventricular Tachycardia (V-Tach), Ventricular Tachycardia (V-Tach), Ventricular Fibrillation (V-FIb), Asystole. The responses to the cardiac status question 640 can be plotted on the cardiac curve 642.

### Conclusion

It should be understood that the data and specific Clinical Warning Curves described herein are intended only as illustrative examples that can be used in implementations of the present disclosure. For example, while five nationally-known content experts in nursing and sepsis were referred to, any number of experts and/or publications can be used to generate data for various Clinical Warning Curves. Additionally, while the present disclosure is described as benefiting newer and less experienced nurses in specific hospital contexts (e.g., preventing sepsis) it should be understood that implementations of the present disclosure can be generalized to any type of healthcare provider under any healthcare setting, in various implementations of the present disclosure.

Failure to Rescue (FTR) poses a significant challenge in healthcare, particularly concerning patient safety and the overall quality of care. New graduate nurses, in particular, face challenges in identifying and managing the early signs of clinical decline, often due to a lack of experience with declining patients, overwhelming workloads, and inadequate staffing. The findings from this study suggest that the implementation of Clinical Warning Curves can be a valuable solution to address this issue. These Curves emerge as valid and reliable tools that aid new graduate nurses in recognizing subtle signs of clinical decline, thus preventing FTR events. By providing a structured framework for identifying and responding to patient complications, the Clinical Warning Curves can enhance the ability of healthcare providers, especially new graduate nurses, to intervene proactively and prevent adverse outcomes.

It should be appreciated that the logical operations described herein with respect to the various figures may be implemented (1) as a sequence of computer-implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special-purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well-known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer-readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

### REFERENCES

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The following patents, applications, and publications, as listed below and throughout this document, describes various application and systems that could be used in combination the exemplary system and are hereby incorporated by reference in their entirety herein.
Barmparas, G., Martin, M. J., Wiegmann, D. A., Catchpole, K. R., Gewertz, B. L., & Ley, E. J. (2016). Increased age predicts failure to rescue. The American Surgeon, 82, 1073-1079.
Considine J., Street, M., Hutchinson, A. M., Mohebbi, M., Rawson, H., Dunning, T., Botti, M., Duke, M. M., Hutchison, A. F., & Bucknall, T. (2020). Vital sign abnormalities as predictors of clinical deterioration in subacute care patients: A prospective case-time-control study. International Journal of Nursing Studies, 108, 1-9. https://doi.org/10.1016/j.ijnurstu.2020.103612
Chua, W. L., Legido-Quigley, H., Ng, P. Y., McKenna, L., Hassan, N. B., & Liaw, S. Y. (2019).
Seeing the whole picture in enrolled and registered nurses' experiences in recognizing clinical deterioration in general ward patients: A qualitative study. International Journal of Nursing Studies, 95, 56-64. https://doi.org/10.1016/j.ijnurstu.2019.04.012
Chua, W. L., Legido-Quigley, H., Jones, D., Hassan, N. B., Tee, A., & Liaw, S. Y. (2020). A call for better doctor-nurse collaboration: A qualitative study of the experiences of junior doctors and nurses in escalating care for deteriorating ward patients. Australian Critical Care, 33, 54-61. https://doi.org/10.1016/j.aucc.2019.01.006
Cull, J., Brevetta, R., Gerac, J., Kothari, S., & Blackhurst, D. (2023). Epic sepsis model inpatient predictive analytic tool: A validation study. Critical Care Explorations, 5 (7), 1-10. https://doi.org/10.1097/CCE.0000000000000941
Dennison, R. D. (2018). Pass CCRN! (5th ed). Mosby. (2015).
Failure to rescue: The nurse's impact. MEDSURG Nursing, 24 (3), 145-149.
M., & Winfield, S. (2022). Rising to the COVID-19 challenge: Creating a remote experiential simulation. Journal for Nurses in Professional Development, 39 (3), E27-E31. ttps://doi.org/10.1097/NND.0000000000000823
Herron, E. K. (2017). New graduate nurses' preparation for recognition and prevention of failure to rescue: A qualitative study. Journal of Clinical Nursing, 27, e390-e401. https://doi.org/10.1111/jocn.14016
Huff, J. (2016). ECG workout: Exercises in arrhythmia interpretation. (7th ed.). Lippincott, Williams, & Wilkins.
Levy, M. M., Evans, L. E., & Rhodes, A. (2018). The surviving sepsis campaign bundle: 2018 update. Intensive Care Med, 44, 925-928.
Lynn, M. R. (1986). Determination and quantification of content validity. Nursing Research, 35 (6), 382-385.
Earl-Royal, E., Kaufman, E. J., Hsu, J. Y., Wiebe, D. J., Reilly, P. M., & Holena, D. N. (2016). Age and preexisting conditions as risk factors for severe adverse events and failure to rescue after injury. Journal of Surgical Research, 205, 368-377. http://dx.doi.org/10.1016/j.jss.2016.06.082
Garvey, P. (2015). Failure to rescue: The nurse's impact. MEDSURG Nursing, 24 (3), 145-149.
Jones, A., & Johnstone, M. J. (2017). Inattentional blindness and failures to rescue the deteriorating patient in critical care, emergency and perioperative settings: Four case scenarios. Australian Critical Care, 30, 219-223. http://dx.doi.org/10.1016/j.aucc.2016.09.005
Kuo, L. E., Kaufman, E., Hoffman, R. L., Pascual, J. L., Martin, N. D., Kelz, R. R., & Holena, D. N. (2017). Failure-to-rescue after injury is associated with preventability: The results of a mortality panel review of failure-to-rescue cases in trauma. Surgery, 161 (3), 782-790. http://dx.doi.org/10.1016/j.surg.2016.08.017
O'Shaughnessy, J., Grzelak, M., Dontsova, A., & Braun-Alfano, I. (2017). Early sepsis identification. MedSurg Nursing, 26 (4), 248-252.
Radigan, K. (2019). Diagnosis sepsis: Is newer better? Critical Care Alert, 26 (10). ISSN: 1067-9502.
Rhodes, A., Evans, L. E., Alhassanis, W., Levy, M. M., Antonellis, M., Ferrer, R., Kumar, A., Sevransky, J. E., Sprung, C. L., Nunnally, M. E., Rochwerg, B., Rubenfeld, G. D., Angus, D. C., Annane, D., Beale, R. J., Bellinghan, G. J., Bernard, G. R., Chiche, J. D., Coopersmith, C., De Backer, D. P., . . . & Dellinger, R. P. (2017). Surviving sepsis campaign: International guidelines for management of sepsis and septic shock: 2016.
Intensive Care Medicine, 43, 304-377. https://doi.org/10.1007/s00134-017-4683-6 Rogers E. M. (2003). Diffusion of innovations. New York, NY: Free Press.
Roussas, A., Masjedi, A., Hanna, K., Zeeshan, M., Kulvatunyou, N., Gries, L., Tang, A., & Joseph, B. (2020). Number and type of complications associated with failure to rescue in trauma patients. Journal of Surgical Research, 254, 41-48. https://doi.org/10.1016/j.jss.2020.04.022
Scantling, D., Hatchimonji, J., Kaufman, E., Xiong, R., Yang, W., & Holena, D. N. (2020). Pulmonary complications in trauma: Another bellwether for failure to rescue? Surgery, xxx, 1-10. https://doi.org/10.1016/j.surg.2020.08.017
Schorr, C. (2016). Nurses can help improve outcomes in severe sepsis. American Nurse Today, 11 (3), 20-24.
Sharoky, C. E., Martin, N. D., Smith, B. P., Pascual, J. L., Kaplan, L. J., Reilly, P. M., & Holena, D. N. (2019). The location and timing of failure-to-rescue events across a statewide trauma system. Journal of Surgical Research, 235, 529-535. https://doi.org/10.1016/j.jss.2018.10.017
Sterner, A., Hagiwara, M. A., Ramstrand, N., & Palmer, L. (2019). Factors developing nursing students' and novice nurses' ability to provide care in acute situations. Nurse Education in Practice, 35, 135-140. https://doi.org/10.1016/j.nepr.2019.02.005
Shankar-Hari, M., Harrison, D. A., Rubenfeld, G. D., & Rowan, K. (2017). Epidemiology of sepsis and septic shock in critical care units: Comparison between sepsis-2 and sepsis-3 populations using a national critical care database. British Journal of Anaesthesia, 119 (4), 626-636. https://doi.org/10.1093/bja/aex234
Sterner, A., Hagiwara, M. A., Ramstrand, N., & Palmer, L. (2019). Factors developing nursing students' and novice nurses' ability to provide care in acute situations. Nurse Education in Practice, 35, 135-140. https://doi.org/10.1016/j.nepr.2019.02.005
Trepanier, S., Yoder-Wise, P., & Finis, N. (2020). From policy to execution: Residency programs for new graduate nurses. Nurse Leader, 18 (2), 172-177.
Vermassen, J., Decruyenaere, J., De Bus, L., Depuydt, P., & Colpaert, K. (2021). Characteristics of sepsis-2 septic shock patients failing to satisfy the sepsis-3 septic shock definition: An analysis of real-time collected data. Ann. Intensive Care, 11 (154), 1-8. https://doi.org/10.1186/s13613-021-00942-1
Weiner, B. J., Lewis, C. C., Stanick, C., Powell, B. J., Dorsey, C. N., Clary, A. S., Boynton, M. H., & Halko, H. (2017). Psychometric assessment of three newly developed implementation outcome measures. Implement Sci., 12 (1), 1-12. https://doi.org/10.1186/s13012-017-0635-3.
Wong, H. J., Bierbrier, R., Ma, P., Quan, S., Sannie, L., & Wu, R. C. (2017). An analysis of messages sent between nurses and physicians in deteriorating internal medicine patients to help identify issues in failures to rescue. International Journal of Medical Informatics, 100, 9-15. http://dx.doi.org/10.1016/j.ijmedinf.2017.01.008
Zegrean, M., Cambridge, B., & Thompson, W. L. (2023). Early recognition of patient deterioration: Application of the modified early warning score. Teaching and Learning in Nursing, 18, 486-491. https://doi.org/10.1016/j.teln.2023.07.008

What is claimed:

1. A computer-implemented method to prevent a failure to rescue event, the method comprising:
providing a user with a first query;
receiving a first user input in response to the first query;
providing the user with a second query, wherein the second query is at least partially based on the first user input;
receiving a second user input in response to the second query;
analyzing at least one of the first user input and the second user input to provide a failure to rescue analysis; and
outputting the failure to rescue analysis.

2. The computer-implemented method of claim 1, wherein analyzing at least one of the first user input and the second user input comprises performing a comparison of the first user input and the second user input to a branching logic database, and wherein outputting the failure to rescue analysis is based on the comparison.

3. The computer-implemented method of claim 1, wherein the failure to rescue analysis comprises a three-point scale.

4. The computer-implemented method of claim 1, wherein the failure to rescue analysis comprises a four-point scale.

5. The computer-implemented method of claim 1, wherein the failure to rescue analysis comprises a treatment recommendation.

6. The computer-implemented method of claim 1, wherein the failure to rescue analysis comprises a predicted diagnosis or a predicted prognosis.

7. The computer-implemented method of claim 1, wherein the failure to rescue analysis comprises an analysis of at least one of: sepsis, trauma, cardiac ectopy, geriatric trauma, respiratory distress, respiratory failure, intracranial hemorrhage, stages of sepsis or cardiac arrest.

8. The computer-implemented method of claim 1, wherein the second query is at least partially based on comparing the first query to a branching logic database.

9. The computer-implemented method of claim 8, wherein the second query is at least partially based on the first query.

10. The computer-implemented method of claim 8, wherein the second query is determined by comparing the first user input to a database of medical reference values.

11. The computer-implemented method of claim 8, further comprising generating graphical display data associated with the failure to rescue analysis.

12. The computer-implemented method of claim 11, wherein the graphical display data comprises color-coded patient statuses plotted on a clinical warning curve.

13. A computer-implemented method for failure to rescue prevention, the method comprising:
receiving an electronic medical record;
performing a failure to rescue analysis by comparing the electronic medical record to a branching logic database; and
outputting the failure to rescue analysis.

14. The computer-implemented method of claim 13, wherein the failure to rescue analysis comprises a three-point scale.

15. The computer-implemented method of claim 13, wherein the failure to rescue analysis comprises a four-point scale.

16. The computer-implemented method of claim 13, wherein the failure to rescue analysis comprises a treatment recommendation.

17. The computer-implemented method of claim 13, wherein the failure to rescue analysis comprises a predicted diagnosis or a predicted prognosis.

18. The computer-implemented method of claim 13, wherein the failure to rescue analysis comprises an analysis of at least one of: sepsis, trauma, cardiac ectopy, geriatric trauma, respiratory distress, respiratory failure, intracranial hemorrhage, stages of sepsis or cardiac arrest.

19. The computer-implemented method of claim 13, wherein the branching logic database comprises a database of medical reference values.

20. A non-transitory computer readable medium having instructions stored therein, wherein execution of the instructions by a processor, causes the processor to:
provide a user with a first query;
receive a first user input in response to the first query;
provide the user with a second query, wherein the second query is at least partially based on the first user input;
receive a second user input in response to the second query;
analyze at least one of the first user input and the second user input to provide a failure to rescue analysis; and
output the failure to rescue analysis.
